(19) United States
(12) Patent Application Publication
(10) Pub. No.: US 2025/0014754 A1
(43) Pub. Date: Jan. 9, 2025

(54) CLINICAL RISK PREDICTION SYSTEM ORIENTED TO DATA DISTRIBUTION DRIFT DETECTION AND SELF-ADAPTATION

(71) Applicant: ZHEJIANG LAB, Hangzhou (CN)

(72) Inventors: Jingsong LI, Hangzhou (CN); Shengqiang CHI, Hangzhou (CN); Feng WANG, Hangzhou (CN); Tianshu ZHOU, Hangzhou (CN); Yu TIAN, Hangzhou (CN)

(21) Appl. No.: 18/635,048

(22) Filed: Apr. 15, 2024

(30) Foreign Application Priority Data
Jul. 4, 2023 (CN) 202310809676.4

(51) Int. Cl. G16H 50/30; G06F 18/2413; G16H 50/20

(52) U.S. Cl. CPC G16H 50/30; G06F 18/24137; G16H 50/20

(57) ABSTRACT

A clinical risk prediction system oriented to data distribution drift detection and self-adaptation, comprising a central server comprising a first drift detection module and a model aggregation module, and nodes comprising a data acquisition module configured to acquire patient clinical diagnosis and treatment data, a second drift detection module and a model updating module. The first and second drift detection module determine whether the patient clinical diagnosis and treatment data distribution has drifted according to whether the new/old patient clinical diagnosis and treatment data set comes from the same data distribution. When the data distribution has drifted, a local clinical risk prediction model is trained, and its parameters are uploaded to the central server and aggregated to obtain an updated model, which is issued to each node for deployment. The new patient clinical diagnosis and treatment data is input into the updated model to obtain a clinical risk prediction result.

### CROSS-REFERENCE TO RELATED APPLICATIONS

The present application claims priority to Chinese Patent Application No. 202310809676.4, filed on Jul. 4, 2023, the content of which is incorporated herein by reference in its entirety.

### TECHNICAL FIELD

The present disclosure belongs to the technical field of medical and health information, in particular to a clinical risk prediction system oriented to data distribution drift detection and self-adaptation.

### BACKGROUND

In the application scenario of clinical risk prediction, demography, disease prevalence, clinical practice and medical care system as a whole may undergo changes over time. The distribution of data will change unpredictably with time, and models established on old data sets are no longer applicable to new data, which means that a clinical risk prediction model based on single-center static cross-sectional data may be outdated or not applicable to other institutions, resulting in inaccurate prediction results. Secondly, the application of the clinical risk prediction model to clinical practice can alter clinical decision-making and intervention measures, causing changes in the outcome distribution for new data and the relationship between predictors and outcomes, thereby leading to a rapid decline in the performance of the clinical risk prediction model. Therefore, the clinical risk prediction model needs to be retrained and deployed after a period of time.

In the scenario of prognosis risk prediction for tumor patients, with advancements in tumor detection methods, the discovery of biomarkers and improvements of treatment methods, the characteristics of clinical diagnosis and treatment data and the distribution of clinical observation outcomes of tumor patients are constantly changing. These factors urge the clinical risk prediction model for tumor prognosis risk assessment to be updated in time as necessary.

Common adaptive updating methods for models include model retraining, model integration in different time windows and incremental learning. Model retraining needs to consume a lot of computing resources and modeling time. Model integration in different time windows needs to maintain a model pool, and to score new data at the same time, which can consume a lot of computational resources. The incremental learning methods have a catastrophic forgetting phenomenon, whereby over time, the model is updated with the latest data, and the newly obtained data often erases previously learned patterns. In addition, model retraining, model integration and incremental learning all need to specify fixed times for model updates, which can lead to the following two situations:
1. When the update interval is too short, not enough new data with diverse distributions has accumulated, resulting in the current model update producing similar results to the previous one, thus wasting system computing resources;
2. When the update interval is too long, too much accumulated new data causes the model update to lag, leading to poor prediction performance for new data.

Therefore, there is an urgent need to propose a clinical risk prediction system to overcome the inaccuracy of clinical risk prediction caused by data drift.

### SUMMARY

In view of the shortcomings of the prior art, the present disclosure provides a clinical risk prediction system oriented to data distribution drift detection and self-adaptation.

According to a first aspect of the embodiment of the present disclosure, a clinical risk prediction system oriented to data distribution drift detection and self-adaptation is provided, which includes a central server and a plurality of nodes.

The central server includes a first drift detection module and a model aggregation module.

The nodes include a data acquisition module, a second drift detection module and a model updating module.

The data acquisition module is used to acquire patient clinical diagnosis and treatment data.

The first drift detection module and the second drift detection module are used to determine whether the patient clinical diagnosis and treatment data has drifted according to whether a new patient clinical diagnosis and treatment data set and an initial patient clinical diagnosis and treatment data set are from a same data distribution.

When a patient clinical diagnosis and treatment data distribution has drifted, a local clinical risk prediction model is trained by the model updating module, parameters of a trained local clinical risk prediction model are uploaded to the central server, the parameters of the local clinical risk prediction model of each node are aggregated by the model aggregation module to obtain an updated clinical risk prediction model, and the updated clinical risk prediction model is issued to each node for deployment; and new patient clinical diagnosis and treatment data are input into the updated clinical risk prediction model to obtain a clinical risk prediction result.

According to a second aspect of the embodiment of the present disclosure, a clinical risk prediction device oriented to data distribution drift detection and self-adaptation is provided, which includes a memory and a processor. The memory is coupled with the processor. The memory is used for storing program data, and the processor is used for executing the program data to implementing the clinical risk prediction system oriented to data distribution drift detection and self-adaptation.

According to a third aspect of the embodiment of the present disclosure, a computer-readable storage medium is provided, on which a computer program is stored, and when executed by a processor, the program implements the above-mentioned clinical risk prediction system oriented to data distribution drift detection and self-adaptation is realized.

Compared with the prior art, the present disclosure has the following beneficial effects.

(1) In the present disclosure, the nodes are configured to communicate only with the central server, and there is no communication among the nodes; at the same time, each node only uploads the parameters of the local clinical risk prediction model to the central server, and does not upload the original patient clinical diagnosis and treatment data set to the central server, so that the present disclosure carries out multi-center data distribution drift detection and multi-center clinical risk prediction model update on the premise of data security and privacy protection.

(2) In the training process of the clinical risk prediction model, the present disclosure determines the weight of the model parameter similarity constraint in the loss function based on the similarity between the new patient clinical diagnosis and treatment data set and the initial patient clinical diagnosis and treatment data set; at the same time, the model parameter similarity constraint refines the knowledge in the old model, which avoids the catastrophic forgetting phenomenon in model update and maintains the accuracy of clinical risk prediction.

(3) When new patient clinical diagnosis and treatment data are generated in the system, the present disclosure can detect the data distribution drift in time. If a data distribution drift is detected, the clinical risk prediction model is updated; if no data distribution drift is detected, the data are saved for the next data distribution drift detection and clinical risk prediction model update. Therefore, the present disclosure can update the clinical risk prediction model after automatically detecting the data distribution drift, without the need to preset a time interval for updating the clinical risk prediction model, which improves the accuracy of clinical risk prediction, and can effectively reduce the waste of computing resources on the premise of timely updating the clinical risk prediction model.

### BRIEF DESCRIPTION OF DRAWINGS

In order to explain the technical solution in the embodiment of the present disclosure more clearly, the drawings necessary for the description of the embodiment will be briefly introduced below. Obviously, the drawings in the following description are only some embodiments of the present disclosure. For those skilled in the art, other drawings can be obtained according to these drawings without any creative work.

FIG. 1 is a schematic diagram of a clinical risk prediction system oriented to data distribution drift detection and self-adaptation provided by an embodiment of the present disclosure;

FIG. 2 is a schematic diagram for determining whether the patient clinical diagnosis and treatment data distribution drifts according to an embodiment of the present disclosure;

FIG. 3 is a schematic diagram of updating a multi-center clinical risk prediction model provided by an embodiment of the present disclosure; and

FIG. 4 is a schematic diagram of a clinical risk prediction device oriented to data distribution drift detection and self-adaptation provided by an embodiment of the present disclosure.

### DESCRIPTION OF EMBODIMENTS

In the following, the technical solution in the embodiment of the present disclosure will be clearly and completely described with reference to the attached drawings. Obviously, the described embodiment is only a part of the embodiment of the present disclosure, but not the whole embodiment. Based on the embodiments in the present disclosure, all other embodiments obtained by those skilled in the art without creative work belong to the scope of protection of the present disclosure.

It should be noted that features in the following embodiments and implementations can be combined with each other without conflict.

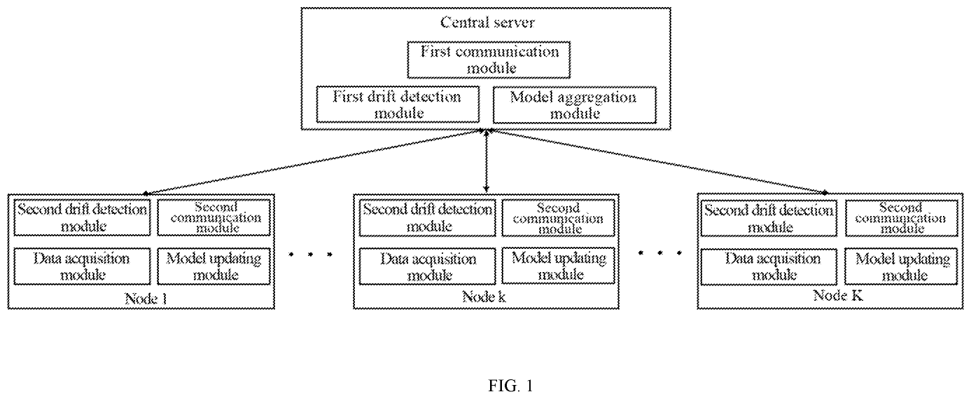

As shown in FIG. 1, the embodiment of the present disclosure provides a clinical risk prediction system oriented to data distribution drift detection and self-adaptation, which includes a central server and a plurality of nodes communicating with the central server.

The central server includes a first drift detection module and a model aggregation module.

The nodes include a data acquisition module, a second drift detection module and a model updating module.

The data acquisition module is used for acquiring and store patient clinical diagnosis and treatment data. The patient clinical diagnosis and treatment data include demographic information, visits, diagnosis, laboratory examination, medical examination, surgery, medication and follow-up information of patients.

The first drift detection module and the second drift detection module are used for determining whether the patient clinical diagnosis and treatment data has drifted according to whether the new patient clinical diagnosis and treatment data set and the initial patient clinical diagnosis and treatment data set are from the same data distribution.

When the patient clinical diagnosis and treatment data distribution drifts, a local clinical risk prediction model is trained by the model updating module, the parameters of the trained local clinical risk prediction model are uploaded to the central server, and the parameters of the local clinical risk prediction model of each node are aggregated by the model aggregation module to obtain an updated clinical risk prediction model, which is then issued to each node for deployment. The new patient clinical diagnosis and treatment data is input into the updated clinical risk prediction model to obtain a clinical risk prediction result.

It should be noted that each node infers the clinical risk of the new patient clinical diagnosis and treatment data by using the local clinical risk prediction model, and its original patient clinical diagnosis and treatment data cannot leave the node. The central server is responsible for detecting the changes of the clinical diagnosis and treatment data distribution with time and updating the clinical risk prediction model. Each node can only communicate with the central server, but not with each other, therefore the present disclosure can carry out multi-center data distribution drift detection and multi-center clinical risk prediction model update on the premise of data security and privacy protection.

The clinical risk prediction system oriented to data distribution drift detection and self-adaptation further includes a first communication module deployed on the central server and a second communication module deployed on the node.

At the initial moment t₀, each node k has an initial patient clinical diagnosis and treatment data set (Xₜ_{₀}^{k}, Yₜ_{₀}^{k}), in which, Xₜ_{₀}^{k} is data features at the moment t₀, Yₜ_{₀}^{k} is a data label at the moment

$${X}_{{t}_{0}}^{k} ∈ {R}^{{N}_{{t}_{0}}^{k} \times D} , {Y}_{{t}_{0}}^{k} ∈ {R}^{{N}_{{t}_{0}}^{k} \times 1} , {N}_{{t}_{0}}^{k}$$

is the sample size of the node k at the moment t₀, and D is the feature number of the data. The model updating module on the node k trains a local clinical risk prediction model clfₜ_{₀}^{k} based on the initial patient clinical diagnosis and treatment data feature Xₜ_{₀}^{k} and a corresponding label Yₜ_{₀}^{k}. The data feature includes multi-source and multi-dimensional information of the patient such as demographics, visits, diagnosis, laboratory examination, medical examination, surgery, medication and follow-up, and the data label may be whether the patient has cardiovascular diseases and other diseases. The clinical risk prediction model is a fully connected neural network.

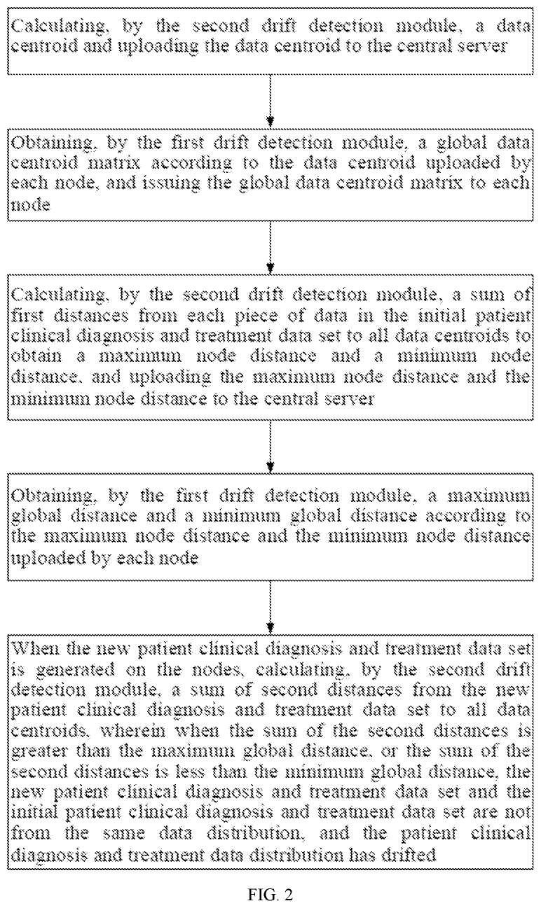

Further, as shown in FIG. 2, the first drift detection module and the second drift detection module determining whether the patient clinical diagnosis and treatment data distribution has drifted according to whether the new patient clinical diagnosis and treatment data set and the initial patient clinical diagnosis and treatment data set are derived from the same data distribution is as follows:

It is supposed that there are K nodes in the clinical risk prediction system oriented to data distribution drift detection and self-adaptation provided by the embodiment of the present disclosure. The process of determining whether the patient clinical diagnosis and treatment data distribution drifts is described by taking the node k as an example, k∈K.

The second drift detection module calculates the data centroid cₜ_{₀}^{k} e of the node k at the moment t₀, cₜ_{₀}^{k}∈R^{D}. The feature value of each dimension of the data centroid cₜ_{₀}^{k} is calculated from the feature of each dimension of the initial patient clinical diagnosis and treatment data set Xₜ_{₀}^{k}. If the features in the initial patient clinical diagnosis and treatment data set Xₜ_{₀}^{k} are categorical variables, the mode of the features in the initial patient clinical diagnosis and treatment data set Xₜ_{₀}^{k} is used as the feature value of the feature corresponding to the data centroid cₜ_{₀}^{k}. If the features in the initial patient clinical diagnosis and treatment data set Xₜ_{₀}^{k} are continuous variables, according to the knowledge of clinical experts, it is determined to use the median or average of the features in the initial patient clinical diagnosis and treatment data set Xₜ_{₀}^{k} as the feature value of the feature corresponding to the data centroid cₜ_{₀}^{k}.

Each node sends the data centroid calculated locally to the central server.

The first drift detection module on the central server obtains a global data centroid matrix Cₜ_{₀} at the moment t₀ according to the data centroid uploaded by each node, Cₜ_{₀}␣R^{K×D}, and sends the global data centroid matrix Cₜ_{₀} to each node through the first communication module.

On the node k, the second drift detection module calculates the sum of the first distances from each data in the initial patient clinical diagnosis and treatment data set Xₜ_{₀}^{k} to all data centroids, and obtains the maximum node distance maxₜ_{₀}^{k} and the minimum node distance minₜ_{₀}^{k}, and uploads them to the central server. In this example, the weighted Euclidean distance is used to calculate the sum of the first distances from each data in the initial patient clinical diagnosis and treatment data set to all data centroids. Because each feature has different importance to the clinical risk prediction model, the relative importance of different features must be considered in the calculation of clinical diagnosis and treatment data distance. According to the knowledge of clinical experts, the distance calculation is carried out by taking the clinical pathological features and treatment solutions which play an important role in clinical risk prediction as high-weight features.

After comparing the maximum and minimum values of each node, the first drift detection module on the central server obtains the global maximum value MAXₜ_{₀} and minimum value MINₜ_{₀} at the moment t₀ and sends them to each node through the first communication module on the central server.

When a new patient clinical diagnosis and treatment data set xₜ^{k} is generated on the node k, the second drift detection module needs to determine whether the new patient clinical diagnosis and treatment data set xₜ^{k} and the initial patient clinical diagnosis and treatment data set Xₜ_{₀}^{k} come from the same data distribution. In an embodiment, the second drift detection module calculates the sum de of the second distances from the new patient clinical diagnosis and treatment data set xₜ^{k} to all data centroids. When the sum of the second distances is greater than the maximum global distance, or the sum of the second distances is less than the minimum global distance (that is, dₜ^{k}>MAXₜ_{₀} or dₜ^{k}<MINₜ_{₀}), it is determined that the new patient clinical diagnosis and treatment data set and the initial patient clinical diagnosis and treatment data set are not from the same data distribution, and the patient clinical diagnosis and treatment data distribution has drifted.

It should be noted that the clinical risk prediction model does not need to be updated if the patient clinical diagnosis and treatment data distribution does not drift; if the patient clinical diagnosis and treatment data distribution drifts, the clinical risk prediction model needs to be updated. After the clinical risk prediction model is updated, the system enters the next update cycle and is at the initial moment of the next update cycle. At this point, all the patient clinical diagnosis and treatment data on the node are the initial patient clinical diagnosis and treatment data set of the node.

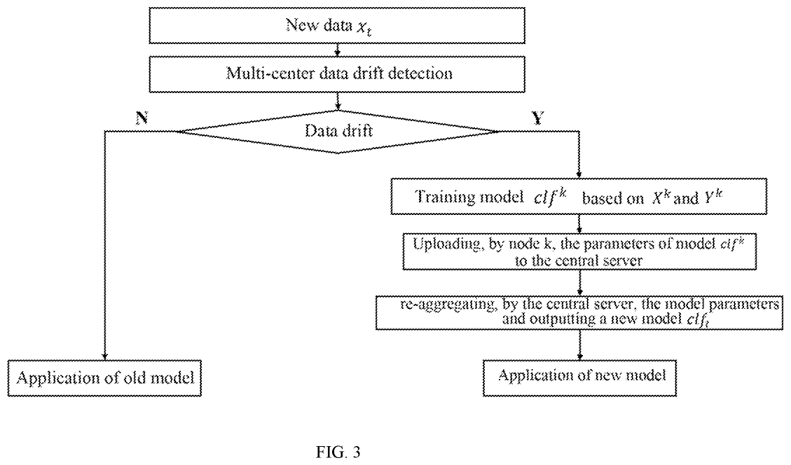

Further, as shown in FIG. 3, when the patient clinical diagnosis and treatment data distribution drifts, a local clinical risk prediction model is trained by the model updating module, the parameters of the trained local clinical risk prediction model are uploaded to the central server, and the parameters of the local clinical risk prediction model of each node are aggregated by the model aggregation module to obtain an updated clinical risk prediction model, which is then issued to each node for deployment. The new patient clinical diagnosis and treatment data are input into the updated clinical risk prediction model to obtain a clinical risk prediction result, which include the following:

Training the local clinical risk prediction model through the model updating module includes the following:

The model updating module trains the local clinical risk prediction model clf^{k} based on a first loss function. The first loss function is the sum of a second loss function l₁^{k}(θₜ) and a third loss function λl₂^{k}(θₜ_{₀}, θₜ). The third loss function λl₂^{k}(θₜ_{₀}, θₜ) is the product of a weight adjustment coefficient λ and a model parameter similarity constraint term l₂^{k}(θₜ_{₀}, θₜ). The second loss function l₁^{k}(θₜ) is a logarithmic loss function between a data label Y^{k} corresponding to all patient clinical diagnosis and treatment data sets at the current moment and a prediction probability P^{k} of the local clinical risk prediction model. The weight adjustment coefficient λ is determined based on the similarity between the initial patient clinical diagnosis and treatment data set and all patient clinical diagnosis and treatment data sets at the current moment.

In an embodiment, the expression of the first loss function is as follows:

$${l}^{k} ( {θ}_{t} ) = {l}_{1}^{k} ( {θ}_{t} ) + λ {l}_{2}^{k} ( {θ}_{{t}_{0}} , {θ}_{t} )$$

In the expression, θₜ_{₀} denotes a parameter of the local clinical risk prediction model trained based on the initial patient clinical diagnosis and treatment data set Xₜ_{₀}^{k} on the node k at the node moment t₀, and θₜ denotes a parameter of the local clinical risk prediction model trained based on all patient clinical diagnosis and treatment data sets X^{k} on the node k at the current moment.

Further, the weight adjustment coefficient λ is determined based on the similarity between the initial patient clinical diagnosis and treatment data set and all patient clinical diagnosis and treatment data sets at the current moment, and the expression is as follows:

$$λ = exp ( - \vert \frac{1}{{N}_{{t}_{0}}^{k}} {\sum }_{i = 1}^{{N}_{{t}_{0}}^{k}} {d}_{{t}_{0}}^{k} - \frac{1}{{N}^{k}} {\sum }_{i = 1}^{{N}^{k}} {d}^{k} \vert )$$

where λ denotes the weight adjustment coefficient, dₜ_{₀}^{k} denotes a sum of distances from each data xₜ_{₀}^{k} in the initial patient clinical diagnosis and treatment data set Xₜ_{₀}^{k} of the node k at the moment t₀ to K data centroids, Nₜ_{₀}^{k} denotes a sample size of the initial patient clinical diagnosis and treatment data set Xₜ_{₀}^{k} of the node k at t₀, d^{k} denotes a sum of distances from each data x^{k} in all patient clinical diagnosis and treatment data sets of the node k at the current moment to K data centroids, and N^{k} denotes the sample size of all patient clinical diagnosis and treatment data sets X^{k} of the node k at the current moment.

Further, the model parameter similarity constraint term is the distance between the first model parameters and the second model parameters. The first model parameters are parameters of the local clinical risk prediction model trained based on the initial patient clinical diagnosis and treatment data set Xₜ_{₀}^{k} on the node k at the moment t₀. The second model parameters are parameters of the local clinical risk prediction model trained based on all patient clinical diagnosis and treatment data sets X^{k} at on the node k the current moment. The expression is as follows:

$${l}_{2}^{k} ( {θ}_{{t}_{0}} , {θ}_{t} ) = {‖ {θ}_{{t}_{0}} - {θ}_{t} ‖}_{1}$$

Each node uploads the parameters of the trained local clinical risk prediction model clf^{k} to the central server through the second communication module. After receiving the local clinical risk prediction model clf^{k}, the central server deletes the old version parameter clfₜ_{₀}^{k} of the local clinical risk prediction model provided by the node k, and aggregates the local clinical risk prediction model clf^{k} of each node with the old version parameter provided by the local clinical risk prediction models of other nodes through the model aggregation module to obtain an updated clinical risk prediction model clfₜ, which is then distributed to each node for deployment.

After receiving the updated clinical risk prediction model clfₜ, each node deploys the clinical risk prediction model, and inputs the new patient clinical diagnosis and treatment data into the updated clinical risk prediction model clfₜ to obtain the clinical risk prediction result.

### Embodiment 1

This embodiment is oriented to the scenario of tumor prognosis risk assessment, and the clinical risk prediction system oriented to data distribution drift detection and self-adaptation will be further elaborated.

Hospital A, Hospital B and Hospital C participate in the construction and application of a local clinical risk prediction model as nodes, and an independent central server D is responsible for communication with the three hospitals. The three hospitals are responsible for collecting clinical diagnosis and treatment data of colorectal cancer patients in their own hospitals, including age, gender, disease diagnosis, complications, blood routine, urine routine, surgical records, drug use records, survival time and survival status.

Hospital A, Hospital B and Hospital C use the clinical diagnosis and treatment data of colorectal cancer patients collected by their respective hospitals, respectively, to construct local clinical risk prediction models based on fully connected neural networks, and obtain local clinical risk prediction models MA, MB and Mc. The three hospitals upload local clinical risk prediction models to the central server D, respectively. The central server D aggregates the parameters of three local clinical risk prediction models to obtain a clinical risk prediction model. Then, the central server D sends the clinical risk prediction model to the three hospitals. The three hospitals deploy the clinical risk prediction model locally and use it to predict the prognosis risk of patients.

During the application of the clinical risk prediction system, the three hospitals will continuously collect the latest clinical diagnosis and treatment data of colorectal cancer patients. The first drift detection module on the central server and the second drift detection module deployed on the node will be responsible for cooperatively detecting whether the clinical diagnosis and treatment data distribution of colorectal cancer patients drifts, which includes the following:

The second drift detection module calculates the data centroid and uploads the data centroid to the central server.

The first drift detection module obtains a global data centroid matrix according to the data centroid uploaded by each node and sends the global data centroid matrix to each node.

The second drift detection module calculates the sum of the first distances from each data in the initial patient clinical diagnosis and treatment data set to the centroids of all data to obtain the maximum and minimum node distances, and uploads them to the central server.

The first drift detection module obtains a maximum global distance and a minimum global distance according to the maximum and minimum node distances uploaded by each node.

When new clinical diagnosis and treatment data of colorectal cancer patients are generated on the node, the second drift detection module calculates the sum of second distances from the clinical diagnosis and treatment data of new colorectal cancer patients to all data centroids; when the sum of the second distances is greater than the maximum global distance, or the sum of the second distances is less than the minimum global distance, the clinical diagnosis and treatment data of new colorectal cancer patients and the initial clinical diagnosis and treatment data of colorectal cancer patients are not from the same data distribution, and the clinical diagnosis and treatment data distribution of colorectal cancer patients has drifted.

If the clinical diagnosis and treatment data distribution of colorectal cancer patients has not drifted, the clinical risk prediction model does not need to be updated; if the clinical diagnosis and treatment data distribution of colorectal cancer patients drifts, the clinical risk prediction model needs to be updated.

The updating of the clinical risk prediction model is carried out under the constraints of the data set similarity and model parameter similarity, including the following:

A local clinical risk prediction model is trained based on a first loss function through a model updating module on a node. The first loss function is the sum of a second loss function and a third loss function. The third loss function is the product of a weight adjustment coefficient and a model parameter similarity constraint term; the second loss function is the logarithmic loss function between the data labels corresponding to all patient clinical diagnosis and treatment data sets at the current moment and the prediction probability of the local clinical risk prediction model. The weight adjustment coefficient is determined based on the similarity between the initial patient clinical diagnosis and treatment data set and all the patient clinical diagnosis and treatment data sets at the current moment.

Corresponding to the aforementioned embodiment of the clinical risk prediction system oriented to data distribution drift detection and self-adaptation, the present disclosure further provides an embodiment of a clinical risk prediction device oriented to data distribution drift detection and self-adaptation.

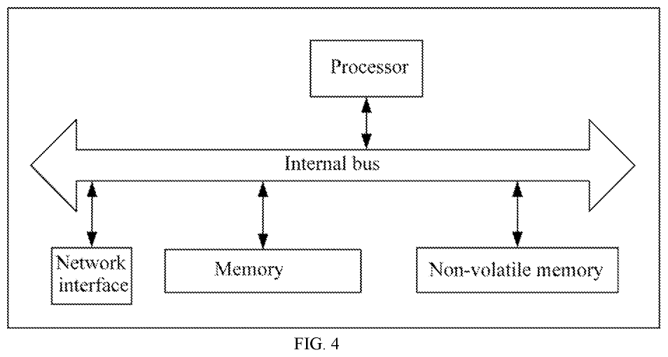

Referring to FIG. 4, a clinical risk prediction device oriented to data distribution drift detection and self-adaptation provided by an embodiment of the present disclosure includes one or more processors for implementing the clinical risk prediction system oriented to data distribution drift detection and self-adaptation in the above embodiment.

The embodiment of the clinical risk prediction device oriented to data distribution drift detection and self-adaptation of the present disclosure can be applied to any equipment with data processing capability, which can be devices or apparatuses such as computers. The embodiment of the device can be realized by software, or by hardware or a combination of hardware and software. Taking the software implementation as an example, as a logical device, it is formed by reading the corresponding computer program instructions in a nonvolatile memory into a memory and running them through the processor of any equipment with data processing capability. From the hardware level, as shown in FIG. 4, it is a hardware structure diagram of any equipment with data processing capability where the clinical risk prediction device oriented to data distribution drift detection and self-adaptation of the present disclosure is located. In addition to the processor, memory, network interface and nonvolatile memory shown in FIG. 4, any equipment with data processing capability where the device is located in the embodiment usually includes other hardware according to the actual functions of the equipment with data processing capability, which will not be described here again.

The realization process of the functions and actions of each unit in the above-mentioned device is detailed in the realization process of the corresponding steps in the above-mentioned method, and will not be repeated here.

For the device embodiment, since it basically corresponds to the method embodiment, it is only necessary to refer to the part of the description of the method embodiment for the relevant points. The device embodiment described above is only schematic, in which the units described as separate components may or may not be physically separated, and the components displayed as units may or may not be physical units, that is, they may be located in one place or distributed to multiple network units. Some or all of the modules can be selected according to actual needs to achieve the purpose of the solutions of the present disclosure. Those skilled in the art can understand and implement the solutions without creative work.

An embodiment of the present disclosure further provides a computer-readable storage medium, on which a program is stored, and when executed by the processor, the program implements the clinical risk prediction system oriented to data distribution drift detection and self-adaptation in the above embodiment.

The computer-readable storage medium can be an internal storage unit of any equipment with data processing capability as described in any of the previous embodiments, such as a hard disk or a memory. The computer-readable storage medium may further be any equipment with data processing capability, such as a plug-in hard disk, a Smart Media Card (SMC), an SD card, a Flash Card and the like. Further, the computer-readable storage medium may further include both internal storage units and external storage devices of any equipment with data processing capability. The computer-readable storage medium is used for storing the computer program and other programs and data required by any equipment with data processing capability, and may further be used for temporarily storing data that has been output or will be output.

Other embodiments of the present disclosure will be easily conceived by those skilled in the art after considering the specification and practicing the disclosure herein. This application is intended to cover any variations, uses or adaptations of this application, which follow the general principles of this application and include common sense or common technical means in this technical field that are not disclosed in this application. The specification and examples are to be regarded as exemplary only.

It should be understood that this application is not limited to the precise structure described above and shown in the drawings, and various modifications and changes may be made without departing from the scope thereof.

What is claimed is:

1. A clinical risk prediction system oriented to data distribution drift detection and self-adaptation comprising:
a central server comprising a first drift detection module and a model aggregation module; and
nodes comprising a data acquisition module, a second drift detection module and a model updating module;
wherein the data acquisition module is configured to acquire patient clinical diagnosis and treatment data;
wherein the first drift detection module and the second drift detection module are configured to determine whether the patient clinical diagnosis and treatment data have drifted according to whether a new patient clinical diagnosis and treatment data set and an initial patient clinical diagnosis and treatment data set are from a same data distribution;
wherein when a patient clinical diagnosis and treatment data distribution has drifted, a local clinical risk prediction model is trained by the model updating module, parameters of a trained local clinical risk prediction model are uploaded to the central server, the parameters of the local clinical risk prediction model of each node are aggregated by the model aggregation module to obtain an updated clinical risk prediction model, and the updated clinical risk prediction model is issued to each node for deployment; and new patient clinical diagnosis and treatment data are input into the updated clinical risk prediction model to obtain a clinical risk prediction result;
wherein said the first drift detection module and the second drift detection module determine whether the patient clinical diagnosis and treatment data have drifted according to whether a new patient clinical diagnosis and treatment data set and an initial patient clinical diagnosis and treatment data set are from a same data distribution comprises:
calculating, by the second drift detection module, a data centroid and uploading the data centroid to the central server;
obtaining, by the first drift detection module, a global data centroid matrix according to the data centroid uploaded by each node, and issuing the global data centroid matrix to each node;
calculating, by the second drift detection module, a sum of first distances from each piece of data in the initial patient clinical diagnosis and treatment data set to all data centroids to obtain a maximum node distance and a minimum node distance, and uploading the maximum node distance and the minimum node distance to the central server;
obtaining, by the first drift detection module, a maximum global distance and a minimum global distance according to the maximum node distance and the minimum node distance uploaded by each node; and
when the new patient clinical diagnosis and treatment data set is generated on the nodes, calculating, by the second drift detection module, a sum of second distances from the new patient clinical diagnosis and treatment data set to all data centroids, wherein when the sum of the second distances is greater than the maximum global distance, or the sum of the second distances is less than the minimum global distance, the new patient clinical diagnosis and treatment data set and the initial patient clinical diagnosis and treatment data set are not from the same data distribution, and the patient clinical diagnosis and treatment data distribution has drifted; and
wherein said when a patient clinical diagnosis and treatment data distribution has drifted, a local clinical risk prediction model is trained by the model updating module comprises:
training, by the model updating module, the local clinical risk prediction model based on a first loss function;
wherein the first loss function is a sum of a second loss function and a third loss function; the third loss function is a product of a weight adjustment coefficient and a model parameter similarity constraint term; the model parameter similarity constraint term is a distance between a first model parameter and a second model parameter; the first model parameter is a parameter of the local clinical risk prediction model trained based on an initial patient clinical diagnosis and treatment data set Xₜ_{ₒ}^{k} on a node k at a moment t₀; and the second model parameter is a parameter of the local clinical risk prediction model trained based on all patient clinical diagnosis and treatment data set X^{k} on the node k at a current moment; and
wherein the second loss function is a logarithmic loss function between data labels corresponding to all patient clinical diagnosis and treatment data sets at the current moment and a prediction probability of the local clinical risk prediction model; and
determining the weight adjustment coefficient based on a similarity between the initial patient clinical diagnosis and treatment data set and all patient clinical diagnosis and treatment data sets at the current moment, with a relational expression as follows:
$$λ = exp ( - \vert \frac{1}{{N}_{{t}_{0}}^{k}} {\sum }_{i = 1}^{{N}_{{t}_{0}}^{k}} {d}_{{t}_{0}}^{k} - \frac{1}{{N}^{k}} {\sum }_{i = 1}^{{N}^{k}} {d}^{k} \vert )$$
where λ denotes the weight adjustment coefficient, dₜ_{₀}^{k} denotes a sum of distances from each piece of data xₜ_{₀}^{k} in the initial patient clinical diagnosis and treatment data set Xₜ_{₀}^{k} of the node k at the moment t₀ to K data centroids, Nₜ_{₀}^{k} denotes a sample size of the initial patient clinical diagnosis and treatment data set Xₜ_{₀}^{k} of the node k at the moment t₀, d^{k} denotes a sum of distances from each piece of data x^{k} in all patient clinical diagnosis and treatment data sets of the node k at the current moment to the K data centroids, and N^{k} denotes a sample size of all patient clinical diagnosis and treatment data sets X^{k} on the node k at the current moment.

2. The clinical risk prediction system oriented to data distribution drift detection and self-adaptation according to claim 1, wherein said calculating, by the second drift detection module, a data centroid comprises:
calculating a feature value of each dimension of the data centroid from features of each dimension of the initial patient clinical diagnosis and treatment data set;
when the features in the initial patient clinical diagnosis and treatment data set are categorical variables, using a mode of the features in the initial patient clinical diagnosis and treatment data set as the feature value of a feature corresponding to the data centroid; and
when the features in the initial patient clinical diagnosis and treatment data set are continuous variables, using a median or an average of the features in the initial patient clinical diagnosis and treatment data set as the feature value of the feature corresponding to the data centroid.

3. The clinical risk prediction system oriented to data distribution drift detection and self-adaptation according to claim 1, wherein said calculating, by the second drift detection module, a sum of first distances from each piece of data in the initial patient clinical diagnosis and treatment data set to all data centroids comprises:
calculating, by using a weighted Euclidean distance, the sum of the first distances from each piece of data in the initial patient clinical diagnosis and treatment data set to all data centroids.

4. The clinical risk prediction system oriented to data distribution drift detection and self-adaptation according to claim 2, wherein the features in the initial patient clinical diagnosis and treatment data set are multi-source and multi-dimensional information comprising demographics, visits, diagnosis, laboratory tests, medical examination, surgery, medication and follow-up information.

5. A clinical risk prediction device oriented to data distribution drift detection and self-adaptation, comprising a memory and a processor, wherein the memory is coupled with the processor, and wherein the memory is configured to store program data, and the processor is configured to execute the program data to implement the clinical risk prediction system oriented to data distribution drift detection and self-adaptation according to claim 1.

6. A computer-readable storage medium on which a computer program is stored, wherein the program, when executed by a processor, is configured to implement the clinical risk prediction system oriented to data distribution drift detection and self-adaptation according to claim 1.
